SEQUENCE LISTING (7 sequences)

SEQ ID NO: 1 - protein, 15 aa, Homo sapiens
MGKKQNRKTGNSKTC

SEQ ID NO: 2 - protein, 32 aa, Homo sapiens
MGKKQNRKTGNSKTQSASPPPKERSSSPATEC

SEQ ID NO: 3 - protein, 147 aa, Homo sapiens
MGKKQNRKTGNSKTQSASPPPKERSSSPATEQSWMENDFDELREEGFRRSNYSELREDIQTKGKEVENFEKNLEECITRITNTEKCLKELMELKTKARELREECRSLRSRCDQLEERVSAMEDEMNEMKREGKFREKRIKRNEQSLC

SEQ ID NO: 4 - protein, 205 aa, Homo sapiens
MGKKQNRKTGNSKTQSASPPPKERSSSPATEQSWMENDFDELREEGFRRSNYSELREDIQTKGKEVENFEKNLEECITRITNTEKCLKELMELKTKARELREECRSLRSRCDQLEERVSAMEDEMNEMKREGKFREKRIKRNEQSLQEIWDYVKRPNLRLIGVPESDVENGTKLENTLQDIIQENFPNLARQANVQIQEIQRTPC

SEQ ID NO: 5 - protein, 259 aa, Homo sapiens
MGKKQNRKTGNSKTQSASPPPKERSSSPATEQSWMENDFDELREEGFRRSNYSELREDIQTKGKEVENFEKNLEECITRITNTEKCLKELMELKTKARELREECRSLRSRCDQLEERVSAMEDEMNEMKREGKFREKRIKRNEQSLQEIWDYVKRPNLRLIGVPESDVENGTKLENTLQDIIQENFPNLARQANVQIQEIQRTPQRYSSRRATPRHIIVRFTKVEMKEKMLRAAREKGRVTLKGKPIRLTADLSAETLC

SEQ ID NO: 6 - protein, 330 aa, Homo sapiens
MGKKQNRKTGNSKTQSASPPPKERSSSPATEQSWMENDFDELREEGFRRSNYSELREDIQTKGKEVENFEKNLEECITRITNTEKCLKELMELKTKARELREECRSLRSRCDQLEERVSAMEDEMNEMKREGKFREKRIKRNEQSLQEIWDYVKRPNLRLIGVPESDVENGTKLENTLQDIIQENFPNLARQANVQIQEIQRTPQRYSSRRATPRHIIVRFTKVEMKEKMLRAAREKGRVTLKGKPIRLTADLSAETLQARREWGPIFNILKEKNFQPRISFPAKLSFISEGERKYFYDKEMLRDFVTTRPTLKELLKEALNMERNNRYC

SEQ ID NO: 7 - protein, 30 aa, Homo sapiens
QMLRDFVTTRPTLKELLKEALNMERNNRYC

(19) United States
(12) Patent Application Publication
(10) Pub. No.: US 2015/0219662 A1
(43) Pub. Date: Aug. 6, 2015

(54) USE OF PROTEIN LINE-1 ORF-1 AS A BIOMARKER FOR CANCER

(71) Applicant: University of Louisville Research Foundation, Inc., Louisville, KY (US)

(72) Inventors: Kenneth S. Ramos, Goshen, KY (US); Saeed A. Jortani, Louisville, KY (US)

(73) Assignee: University of Louisville Research Foundation, Inc., Louisville, KY (US)

(21) Appl. No.: 14/409,671

(22) PCT Filed: Jun. 28, 2013
(86) PCT No.: PCT/US2013/048411

§ 371 Date: Dec. 19, 2014

Related U.S. Application Data
(60) Provisional application No. 61/665,673, filed on Jun. 28, 2012.

(51) Int. Cl. G01N 33/574

(52) U.S. Cl. CPC G01N 33/57496; G01N 33/57434; G01N 2333/47

(57) ABSTRACT

Embodiments of the present invention relate to the detection and diagnosis of cancer. More particularly, the present invention relates to a method for the early detection and diagnosis of cancer by measuring the amount of LINE-1 ORF-1 protein in a liquid biological sample obtained from a human.

### CROSS REFERENCE TO RELATED APPLICATION

This application claims the benefit of priority to U.S. Provisional Patent Application Ser. No. 61/665,673, filed Jun. 28, 2012, for USE OF PROTEIN LINE-1 ORF-1 AS A BIOMARKER FOR CANCER, incorporated herein by reference.

### GOVERNMENT RIGHTS

This invention was made with government support under Grant RO1 ES017274 awarded by the National Institute of Health. The government has certain rights in the invention.

### FIELD OF THE INVENTION

Embodiments of the present invention relate to the detection and diagnosis of cancer. More particularly, the present invention relates to a method for the early detection and diagnosis of cancer by measuring the amount of LINE-1 ORF-1 protein in a liquid biological sample obtained from a human.

### BACKGROUND

Cancer is a significant public health hazard and the second leading cause of death in the United States. Early detection of cancer markedly increases patients' survival rates.

Long Interspersed Nuclear Element-1 (“LINE-1”) is a mammalian retrotransposon that has been linked to several diseases in humans, including cancer. LINE-1 comprises 15 percent to 30 percent of the human and murine chromosomal DNA content. LINE-1 is transcribed from its 5′ UTR (5′ untranslated region) and inserted into the host genome via a copy and paste mechanism that involves an RNA intermediate and reverse transcriptase activity. A complete cycle of retrotransposition can be associated with DNA inversions, duplications or insertions. Several LINE-1 insertion-associated diseases have been identified, most notably hemophilia and colon cancer. Non-insertional mechanisms may also contribute to changes in gene expression via regulation of splicing events leading to production of aberrant RNA products and regulation of gene expression.

LINE-1 is recognized as the most active and only autonomous mobile element in humans. The dicistronic LINE-1 transcript serves as mRNA for the synthesis of two proteins, ORF-1 and ORF-2, in the cytoplasm, as transposition intermediates in the nucleus, and as microRNAs. The multiplicity of LINE-1 functions reflects its central role in cellular homeostasis, and explains why cells have evolved regulatory mechanisms to maintain LINE-1 expression in check. ORF-1 and ORF-2 proteins are used to complete cycles of retrotransposition leading to reinsertion of LINE-1 into the genome. Somatic LINE-1 insertions have been found in the second intron of the myc locus in human breast carcinoma and in the last exon of APC (Adenomatous Polyposis Coli) in a patient with colorectal cancer.

Expression of LINE-1 in normal somatic cells is for the most part undetectable due to DNA methylation, since the large majority of 5-methylcytosine in the genome lies within repetitive sequences, including the CpG islands of LINE-1 elements. Hypomethylation of LINE-1 promoter is associated with increases in LINE-1 expression in several cancers, including breast, testicular, renal, prostate, hepatocellular, chronic lymphocytic leukemia, chronic myeloid leukemia, ovarian carcinomas, and colorectal. The mechanisms that control LINE-1 methylation are poorly understood, but recent evidence has shown that chemical carcinogens and inducers of oxidative stress induce hypomethylation of the LINE-1 promoter to reactivate LINE-1 expression in somatic cells.

Activation (de-repression) of LINE-1 has been implicated in gene mutations, changes in gene expression and induction of DNA damage. Overexpression of LINE-1 in cultured cells induces DNA double strand breaks (DSBs) and is highly cytotoxic. Deletion of the MAEL gene leads to a dramatic increase in endogenous LINE-1 expression and accumulation of large number of double strand breaks throughout the genome in the male mouse germ line.

### SUMMARY

The overexpression of LINE-1 in cells poses a threat to genomic integrity and is associated with increased rates of cellular death. As such, the products of LINE-1 transcription, which are normally absent in somatic cells, may be released into plasma where they accumulate to reflect the inherent activity of LINE-1 in target cells. On the basis of this relationship, the inventors demonstrate here that detection of LINE-1 ORF-1 in plasma serves as a measure of the neoplastic status of the host. This interpretation is in keeping with previous reports showing that patients with cancer have increased levels of circulating DNA compared to healthy volunteers and this DNA is largely of tumor origin, and that LINE-1 methylation status in DNA extracted from the plasma of patients with advanced solid tumors can be used as a biomarker of the pharmacological activity of DNA methylation inhibitors.

The invention disclosed herein provides a quantitative, analytically sensitive, and minimally invasive measurement for early detection of deficits in cellular differentiation and proliferation, as seen in human cancer, by measurement of ORF-1 protein in liquid samples derived from human patients, such as plasma, serum, or urine. Protein ORF-1 serves as a novel biomarker for diagnosis of multiple types of human cancers.

Protein-based diagnostic tools provide a significant advantage over DNA-based diagnostic tools. Peptides may be routinely measured as a part of clinical care of patients for diagnosis, prognosis, and monitoring of patients. In contrast, DNA analysis requires the use of a high complexity laboratory and assay results may not be available for several days.

One aspect of the present invention pertains to a method for the diagnosing LINE-1 overexpression-linked cancer, the method comprising determining the level of ORF-1 protein in a biological sample from a human patient suspected of having cancer, wherein elevated levels of ORF-1 protein relative to a normal control is indicative of cancer. In some embodiments, the biological sample is a liquid sample. In further embodiments, the liquid sample is one of plasma, serum, and urine. In certain embodiments, the liquid sample is one of plasma and serum.

In some embodiments of this aspect, determining the level of ORF-1 protein is performed using an immunoassay. In further embodiments, determining the level of ORF-1 protein includes measuring the binding of ORF-1 to a specific binding agent for ORF-1. In certain embodiments, the specific binding agent for ORF-1 is an antibody specific for ORF-1. In further embodiments, the antibody is configured to bind at least one of SEQ ID NO. 1, SEQ ID NO. 2, SEQ ID NO. 4, SEQ ID NO. 4, SEQ ID NO. 5, SEQ ID NO. 6, and SEQ ID NO. 7.

Another aspect of the present invention pertains to a kit comprising at least one specific binding agent for ORF-1 protein and auxiliary reagents for measurement of ORF-1 protein in at least one of a plasma sample and a serum sample from a human patient. In some embodiments, the kit further comprises instructions for comparing a measured concentration of ORF-1 to specified predetermined concentrations of ORF-1 to determine risk of LINE-1 overexpression-linked cancer in the human patient. In further embodiments, the at least one specific binding agent for ORF-1 protein is an antibody specific for ORF-1 protein.

Yet another aspect of the present invention pertains to a method for the diagnosis of cancer comprising (a) providing a liquid sample obtained from a human, (b) directly contacting the liquid sample with an antibody specific for ORF-1 protein under conditions whereby a complex is formed between the antibody and ORF-1, (c) measuring the amount of complex formed, and (d) comparing the amount of complex formed to a control amount determinative of the diagnosis of cancer. In some embodiments, the liquid sample is one of serum, plasma, and urine. In further embodiments, the antibody specific for ORF-1 protein is one of a monoclonal antibody and a polyclonal antibody. In certain embodiments, the antibody specific for ORF-1 protein is configured to bind at least one of SEQ ID NO. 1, SEQ ID NO. 2, SEQ ID NO. 4, SEQ ID NO. 4, SEQ ID NO. 5, SEQ ID NO. 6, and SEQ ID NO. 7.

Still another aspect of the present invention pertains to a method of detecting prostate cancer comprising (a) providing a biological sample obtained from a human, (b) directly contacting the biological sample with an antibody specific for ORF-1 protein under conditions whereby a complex is formed between the antibody and ORF-1, (c) measuring the amount of complex formed, and (d) comparing the amount of complex formed to a control amount determinative of the diagnosis of prostate cancer. In some embodiments, the biological sample is obtained from a human having a blood concentration of prostate-specific antigen between about 4 ng/ml and about 14 ng/ml. In further embodiments, the biological sample is one of serum, plasma, and urine.

It will be appreciated that the various methods and kits described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings, wherein:

FIG. 1 is a graph showing a standard curve for ELISA measurements of LINE-1 ORF-1 in plasma used to generate the graph in FIG. 2. The vertical axis represents absorbance measured at 450 nm. The horizontal axis represents concentration of ORF-1 measured in ng/ml.

FIG. 2 is a graph comparing average LINE-1 ORF-1 concentrations in plasma of normal patients (left column) and leukemia/breast cancer patients (right column). The vertical axis represents the average concentration of ORF-1 measured in ng/ml. The vertical line in each column represents the standard deviation of the measured concentrations.

FIG. 3 is a graph showing a standard curve for ELISA measurements of LINE-1 ORF-1 in serum used to generate the graphs in FIGS. 4 and 5. The vertical axis represents absorbance measured at 450 nm. The horizontal axis represents concentration of ORF-1 measured in ng/ml.

FIG. 4 is a graph comparing average LINE-1 ORF-1 concentrations in serum from patients with prostate cancer, as confirm by biopsy (left column), and patients where no cancer is suspected (right column). The vertical axis represents the average concentration of ORF-1 measured in ng/ml. The vertical line in each column represents the standard deviation of the measured concentrations.

FIG. 5 is a graph comparing average LINE-1 ORF-1 concentrations in serum from patients with prostate cancer, as confirm by biopsy (left column), and patients where no cancer is suspected (right column). The vertical axis represents the average concentration of ORF-1 measured in ng/ml. The vertical line in each column represents the standard deviation of the measured concentrations.

### DESCRIPTION OF THE PREFERRED EMBODIMENT

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates. At least one embodiment of the present invention will be described and shown, and this application may show and/or describe other embodiments of the present invention. It is understood that any reference to “the invention” is a reference to an embodiment of a family of inventions, with no single embodiment including an apparatus, process, or composition that should be included in all embodiments, unless otherwise stated. Further, although there may be discussion with regards to “advantages” provided by some embodiments of the present invention, it is understood that yet other embodiments may not include those same advantages, or may include yet different advantages. Any advantages described herein are not to be construed as limiting to any of the claims.

References to ORF-1 or ORF-1 protein should be understood to refer to LINE-1 ORF-1 protein. The terms “marker” or “biomarker” are used to indicate that an increased level of ORF-1 protein as measured in a liquid biological sample from a human is indicative of the presence of cancer, specifically LINE-1 overexpression-linked cancer, in that individual. LINE-1 overexpression is known to be linked to at least prostate cancer, leukemia, and breast cancer, and may be linked to additional types of cancer.

As obvious to the skilled artisan, the present invention should not be construed to be limited to the full length LINE-1 ORF-1 protein. Detecting and determining the levels of physiological or artificial fragments of ORF-1 are also encompassed by the present invention. Preferably, any fragments of ORF-1 include at least one epitope of diagnostic interest. Preferably, the epitope exhibits minimal cross reactivity against other known human peptides or proteins. Seven selected epitopes of continuous amino acid sequences within ORF-1, SEQ ID NO. 1-7, are presented in the Sequence Listing section. These sequences are predicted to exhibit minimal cross reactivity against other known human peptides or proteins. In other embodiments, subsections of these epitopes or other epitopes of the ORF-1 protein or fragment thereof may be used as targets for ORF-1 specific binding agents.

In one embodiment, the novel biomarker ORF-1 is used to diagnose cancer. This diagnostic method is based on a liquid sample derived from a human patient. In some embodiments, the liquid sample is plasma, serum, or urine. For measurement, the liquid sample is incubated with an ORF-1 specific binding agent, such as an antibody specific to ORF-1, under conditions appropriate for formation of a binding agent-ORF-1 complex. Such conditions need not be specified, since the skilled artisan can easily identify appropriate incubation conditions without any inventive effort. Similarly, the skilled artisan is well aware of method of generating an antibody with high affinity for its target molecule.

As a final step, the amount of complex is measured and correlated to a diagnosis of cancer. A skilled artisan is aware that there are multiple methods to measure specific binding agent-ORF-1 complex.

In some embodiments, ORF-1 protein is detected using an immunoassay, such as, for example, a competitive enzyme-linked immunosorbent assay (“ELISA”) assay. Briefly, an unlabeled primary antibody specific to ORF-1 is coated onto the wells of a microtiter plate. Unlabeled standards and patient liquid samples are loaded into different wells in the microtiter plate and allowed to incubate with the primary antibody. After this reaction reaches equilibrium, conjugated peptide antigen corresponding to a fragment of ORF-1 protein is added. Primary antibody not already bound to unlabeled antigen, i.e., the standards and samples, will bind the conjugated antigen. Therefore, the more ORF-1 in the samples, the lower the amount of bound conjugated antigen. Secondary antibody including a color change functionality and configured to bind the conjugated antigen is added, then the plate is then developed with substrate and color change is measured. A greater color change indicates a lower concentration of ORF-1. In one embodiment, the conjugated peptide antigen is conjugated to Bovine Serum Albumin (“BSA”) and the secondary antibody is directed against BSA.

In further embodiments, ORF-1 protein is detected using a different competitive ELISA assay. In these embodiments, a peptide antigen corresponding to a fragment of ORF-1 is conjugated to biotin, and the conjugated antigen is coated onto the wells of a microtiter plate that has been pre-coated with streptavidin. The primary antibody specific to ORF-1 is separately incubated with unlabeled standards and patient samples. Once the reaction reaches equilibrium, the primary antibody is added to the microtiter plate. The primary antibody will bind to anchored conjugate wherever the binding sites of the primary antibody are not already occupied by unlabeled antigen, e.g., the samples and standards. Therefore, the more ORF-1 in the patient samples, the lower the amount of primary antibody bound to anchored conjugate. Secondary antibody directed to the primary antibody and including a color change functionality is added, then the plate is then developed with substrate and color change is measured. A greater color change indicates a lower concentration of ORF-1.

In other embodiments, ORF-1 protein is detected using a sandwich ELISA assay. Briefly, an unlabeled primary (capture) antibody specific to a first ORF-1 epitope is coated onto the wells of a microtiter plate. The wells of the plate can optionally be pre-coated with streptavidin, in which case a biotinylated primary antibody would be used. Unlabeled standards and patient liquid samples are loaded into different wells in the microtiter plate and allowed to incubate with the primary antibody. After this reaction reaches equilibrium, a secondary antibody specific to a second ORF-1 epitope is added. The secondary antibody binds ORF-1 protein already bound to the primary antibody. A tertiary (detection) antibody directed against the secondary antibody is then added. The tertiary antibody includes a color change functionality. A stop buffer may be used to stop the reaction once a desired color change has taken place. In certain embodiments, the first ORF-1 epitope and the second ORF-1 epitope are spaced-apart subsets of SEQ ID NO. 1-6. In further embodiments, the first ORF-1 epitope is one of SEQ ID NO. 1-3, and the second ORF-1 epitope is SEQ ID NO. 7.

The antibodies used in the above assays may be polyclonal or monoclonal. Methods for producing and obtaining polyclonal and monoclonal antibodies are known to those skilled in the art. Means for including a color change functionality are known to those skilled in the art. One exemplary means for including a color change functionality with an antibody is to conjugate the antibody to horseradish peroxidase (“HRP”), an enzyme capable of reacting with colorimetric substrates, such as 3,3′,5,5′-tetramethylbenzidine (“TBM”), to produce a detectable color change in the substrates. Other means for including a color change functionality are known to those skilled in the art.

Diagnostic regents in the field of specific binding assays, like ELISA assays, usually are best provided in the form of a kit. The kit comprises at least one specific binding agent, preferably a polyclonal or monoclonal antibody specific to ORF-1, and the auxiliary reagents required to perform the assay. In certain embodiments, the kit is an immunological kit for a competitive ELISA assay and comprises at least one specific binding agent for ORF-1, at least one conjugate ORF-1 or ORF-1 fragment, and auxiliary reagents for measurement of ORF-1 concentration in a liquid sample. Alternatively, the kit is an immunological kit for a sandwich ELISA assay and comprises two specific binding agents for ORF-1 and auxiliary reagents for measurement of ORF-1 concentration in a liquid sample.

Utility of the novel biomarker ORF-1 has been assessed as described in Example 1. The following example and figures are provided to aid the understanding of the present invention, and it is understood that modification can be made in the procedures set forth without departing from the spirit of the invention.

### Example 1

This competitive ELISA assay utilizes custom-made, commercially-produced conjugate and antibody to accurately quantify ORF-1 protein in human plasma. In alternative embodiments, human serum or urine may be used. A peptide corresponding to an ORF-1 epitope is conjugated with PEG-biotin and used as an anchor in a streptavidin coated 96-well plate. A primary antibody which is made against the ORF-1 epitope is introduced, along with a plasma sample. A secondary antibody (GAR-HRP) will then bind to any primary antibody that has not formed a complex with the ORF-1 peptide. A colorimetric substrate will then bind to any GAR-HRP antibody, producing a blue color. A stop buffer which consists of a strong acid will stop the reaction after the desired effect (color) has taken place. Absorbance values are then recorded at 450 nm and patient sample concentrations are calculated based upon a logistic 4-parameter standard curve generated from calibrators of known concentrations.

Storage characteristics; Specimen Handling; and Specimen Stability:

Whole blood is collected in a K₃ EDTA vacutainer tube and is centrifuged at 2500 rpm to separate plasma from white and red cells. In an alternative embodiment where a serum sample is used, whole blood is collected in a serum separator tube and is centrifuged at 2500 rpm to separate serum from white and red cells. In either embodiment, the sample is then transferred to a separate 4 mL vacutainer tube and stored at −20° C. Before analysis can begin the plasma or serum sample is thawed to room temperature and centrifuged at 3600 g. This clean sample is then transferred to a glass tube and ready for analysis.

Specimen Limitations:

Samples must be thawed at room temperature and not be subjected to heat from a heating block or water bath. Samples must also be centrifuged before analysis as particulate matter will negatively affect accuracy and precision of the ELISA.

Reagents:

1. Phosphate Buffered Saline pH 7.4: Prepared by adding 1 packet of PBS powder (Sigma Cat #: P3813) with 1 liter of diH₂O. Stored at room temperature.

2. Phosphate Buffered Saline pH 7.4 with 0.05% Tween 20: Prepared by adding 1 packet of PBS powder (Sigma Cat#: P3813) with 1 liter of diH₂O, then mixing in 500 μL of Tween 20 (Fisher Cat#: BP337-500). Stored at room temperature.

3. Pooled Plasma: Purchased from Biological Specialty Corp. Requested 15 mL aliquots and stored at −80° C. and allowed to thaw at 4° C. one day prior to analysis. The pooled plasma contains the anticoagulant K₃ EDTA and is collected from multiple donors of both genders and has been screened to ensure non-smokers, no viral infections and drug free. Serum is isolated from whole blood collected in serum separator tubes as described above. Aliquots stored at −80° C. and allowed to thaw at 4° C. one day prior to analysis. Urine is collected from remnants of 24-hour collections submitted to clinical laboratory from non-smoker and ostensibly healthy individuals.

4. Biotin-labeled Conjugate (50 ng/mL): A custom made conjugate was prepared by New England Peptide LLC to meet specifications. A peptide with the sequence H₂N-MGKKQNRKTGNSKTC-amide (m.w.=1680) (SEQ ID NO. 1) was coupled to PEG-biotin. A total of 3 mgs were shipped in 1 mg/mL PBS solution. Upon arrival the conjugate was aliquoted into 10 μL volumes and stored at −20° C. A 1 μg/mL working stock was prepared from these aliquots by diluting 1:100 with PBS (2 μL stock+198 μL PBS). The final solution is then made by diluting 60 μL of the 1 μg/mL working stock into 11.940 mL of PBS for a 50 ng/mL solution.

5. Non-labeled Antigen (1 μg/mL): A custom made non-labeled antigen was synthesized by New England Peptide LLC to meet specifications. The peptide has the sequence of H₂N-MGKKQNRKTGNSKTC-amide (m.w.=1680) (SEQ ID NO. 1). A total of 5.5 mgs were shipped and then corrected to a 5 mg/mL stock solution in PBS, this stock was aliquoted into 3 μL volumes and stored at −20° C. This frozen stock is thawed at room temperature, then diluted 1:5 to a 1 mg/mL working stock in PBS (2 μL stock+8 μL PBS). The 1 mg/mL stock is diluted 1:1000 in PBS (3 μL working stock+3 mL PBS) to make a 1 μL/mL final stock that is used to prepare standards and quality controls.

6. Primary Antibody (1:4K): A custom made polyclonal antibody was produced by New England Peptide LLC to meet specifications with a sequence of H₂N-MGKKQNRKTGNSKTC-amide (SEQ ID NO. 1). After immunization of two animals with the conjugated peptide, three separate bleeds were harvested from each animal. Two of the bleeds were combined and then taken through an affinity purification process, as well as the separate bleeds. The final bleed was determined to have a concentration of 0.137 mg/mL, the combined bleeds were determined to have a concentration of 0.224 mg/mL. This combined bleed is used as the primary antibody for this assay. Upon arrival the purified antibody was aliquoted into 5 μL volumes and stored at −20° C. The antibody is diluted 1:4K (3 μL frozen stock+12 μL PBS) to be used in the assay.

7. Secondary Antibody (GAR-HRP): Goat Anti-Rabbit Horseradish Peroxidase whole molecule immunoglobulin was purchased from Sigma-Aldrich (Cat#: A-6667). The 1 mL volume was aliquoted into 3 μL volumes and stored at −20° C. A 1:4K solution is used in this assay (3 μL+12 mL PBS).

8. Color Substrate Reagent: TMB Super Sensitive One Component HRP microwell substrate was purchased from BioFX Laboratories Inc. (Cat#: TMBS-0100-01) and stored at 4° C.

9. Stop Buffer: 2 N HCl (9 mL HCl+41 mL H2O). Concentrated HCl purchased from Sigma-Aldrich (Cat#: H-7020)

10. Streptavidin coated 96-well plates: Plastic 96 well plates pre-coated with streptavidin and blocked with bovine serum albumin. Purchased from Fisher Scientific (Cat#:11-734-776-001) and stored at 4° C.

11. Calibrators: Assay calibrators (standards) are prepared fresh daily and require the 1 μg/mL antigen stock (See Reagent Item#4) as well as a minimum of 15 mL of clean, freshly centrifuged, K₃ EDTA pooled plasma (See Reagent Item#3). Standards are made in 2 mL volumes each of plasma in 13 mm glass tubes. The seven standards have a concentration of 0 (a blank), 1, 2, 5, 7, 10, and 20 ng/mL. They are prepared by spiking 2, 4, 10, 14, 20, and 40 μL of the 1 μL/mL antigen stock into 2 mL of plasma, respectively. Each standard is then diluted (1:1) by transferring 1 mL of spiked plasma into a fresh 13 mm glass tube containing 1 mL of PBS. Any remaining standard is transferred to a 1.5 mL micro-centrifuge tube and stored at −20° C. for stability studies.

12. Quality Controls: Assay quality controls (QCs) are prepared fresh daily and require the 1 μg/mL antigen stock (See Reagent Item#4) as well as a minimum of 7 mL of clean, freshly centrifuged, K₃ EDTA pooled plasma (See Reagent Item#3). QCs are made in 2 mL volumes each of plasma in 13 mm glass tubes. The three QCs have a concentration of 1.0, 2.5, and 5.0 ng/mL. They are prepared by spiking 2.0, 5.0, 10.0 μL of the 1 μL/mL antigen stock into 2 mL of plasma, respectively. Each QC is then diluted (1:1) by transferring 1 mL of spiked plasma into a fresh 13 mm glass tube containing 1 mL of PBS. Any remaining QC is transferred to a 1.5 mL micro-centrifuge tube and stored at −20° C. for stability studies.

Instrumentation:

Amerex Instruments Gyromax 703 Orbital Incubator Shaker (set at 37° C., 100 rpm); Wallac Victor² 1420 Multilabel Counter (set at 450 nm); plate washer; 96-well plate reader; Tecan automated sample processor (Evo Freedom).

Procedure:

1. Centrifuge thawed plasma at 3600×g for 10 min. Remove supernatant for Cal/QC preparation.

2. Prepare Biotin-Labeled Conjugate solution (See Reagents section above, Item#3).

3. Coat 96-well Streptawell plate with 100 μL of conjugate solution per well

4. Incubate the plate at room temperature (20-25° C.) for 1 hour. Use this time to prepare standards, QCs, and samples.

5. Prepare standards (See Reagents section above, Item#10). Prepare QC's (See Reagents section above, Item #11). Prepare subject samples by diluting the sample with an equal volume of PBS (1:1) a minimum volume of 150 μL is required for each subject sample.

6. Wash the plate with 110 μL of PBS-T (using 12-channel pipette) with 5 minute soaks at room temperature, 3×'s. Use this time to prepare the primary antibody solution.

7. Prepare primary antibody solution (1:4K) (See Reagents section above, Item#6).

8. Add 50 μL of calibrators, QCs, and patient samples to their corresponding wells in triplicate.

9. Add 50 μL of primary antibody to each well (except the Blank, add PBS).

10. Incubate the plate for 75 minutes at 37° C. with shaking at 100 rpm.

11. Wash the plate with 110 μL of PBS-T (using 12-channel pipette) with 5 minute soaks at room temperature, 3×'s. Use this time to prepare the secondary antibody solution.

12. Prepare secondary antibody solution (1:4K) (See Reagents section above, Item#7).

13. Add 100 μL of secondary antibody to each well.

14. Incubate the plate for 2 hours at room temperature (20-25° C.).

15. Wash the plate with 110 μL of PBS-T (using 12-channel pipette) with 5 minute soaks at room temperature, 3×'s. During this time remove 11 mL of TMB solution from its bottle and transfer to a 15 mL tube and place in a dark area to allow it to warm to room temperature.

16. Add 100 μL of TMB substrate to each well, incubate at room temperature on a nutator for 7 minutes (or until color is a bright blue).

17. Stop the reaction by adding 100 μL of 2 N HCl to each well.

18. Read the plate using the Wallac Victor² at 450 nm.

19. Export raw data and generate a Logistic 4-parameter curve based upon the experimental standards. Subject sample concentrations are calculated based upon this standard curve.

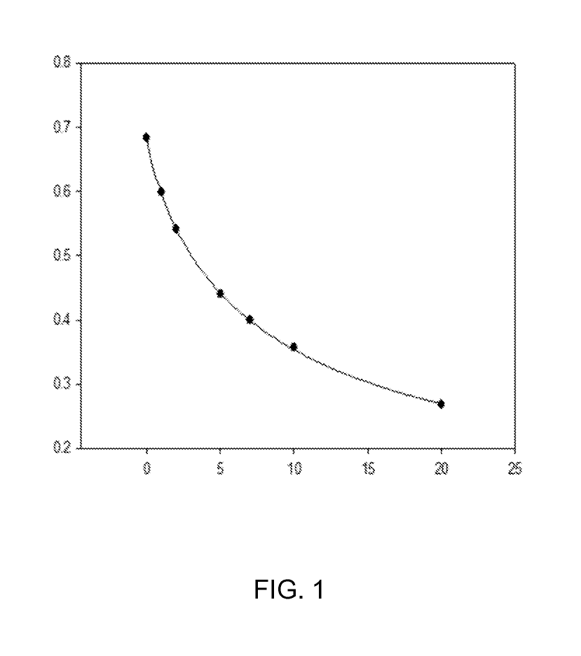

FIG. 1 is a standard curve for ELISA Measurements of ORF-1 in plasma. Seven standards ranging from 0-20 ng/mL ORF-1 are used to construct the curve. Conjugated peptide antigens compete with standard ORF-1 samples for binding sites on the primary antibody. Therefore, the more ORF-1 in the samples, the lower the amount of bound conjugated antigen. Secondary antibody directed to the conjugated antigen and including a color change functionality is utilized, such that a greater color change indicates a lower concentration of ORF-1 as indicated by measured absorbance at 450 nm.

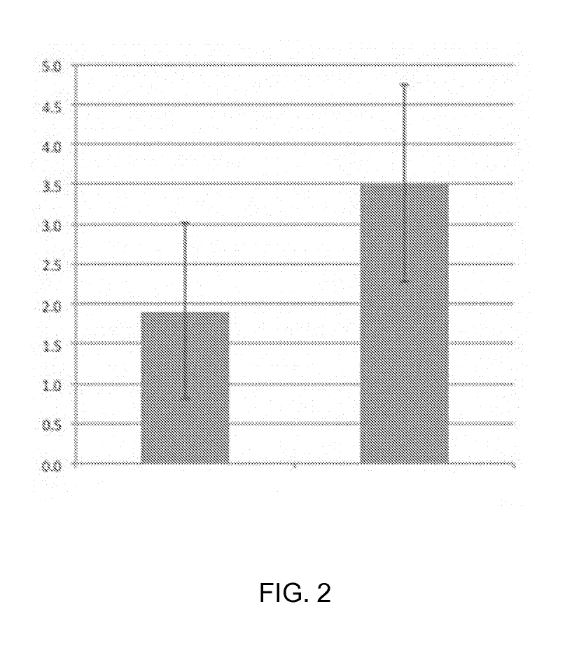

FIG. 2 is a chart showing average ORF-1 concentrations in plasma of normal versus leukemia/breast cancer (abnormal) patients. Plasma collected from patients with known diagnosis of cancer (leukemia or breast) is stored in the refrigerator for up to two days until analysis. Analysis was performed by a competitive ELISA assay similar to that in Example 1, including the patient samples in the same ELISA run as test samples. This data indicates that the cancer group has increased concentrations of ORF-1 protein in their plasma. Contrariwise, results that an unknown plasma sample has increased concentrations of ORF-1 protein serves as a biomarker for the presence of cancer.

In certain embodiments, the specification discloses a method for detecting prostate cancer. Prostate cancer is often diagnosed by determining the level of prostate-specific antigen (“PSA”) protein in a patient's blood. The higher the PSA level, the more likely it is that prostate cancer is present, although factors other than cancer can also result in an elevated PSA level. Blood PSA levels in the range of about 4 ng/ml to about 14 ng/ml are considered a “grey zone;” elevated in comparison to the average healthy patient, but not high enough to strongly indicate the presence of prostate cancer. Detection of PSA levels in the “grey zone” poses a quandary for physicians, as no clear diagnosis can be made from the results.

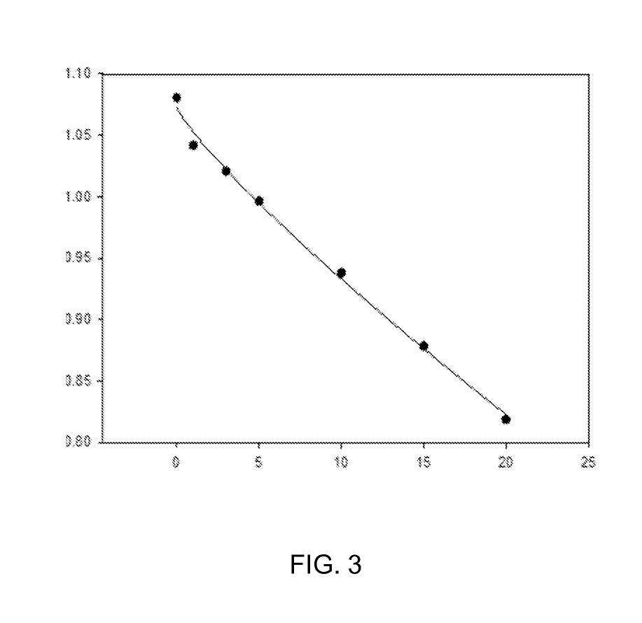

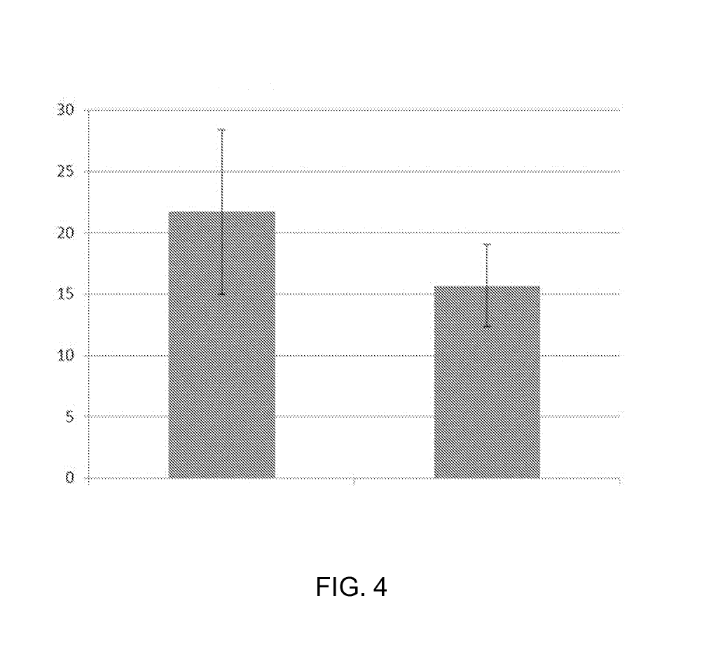

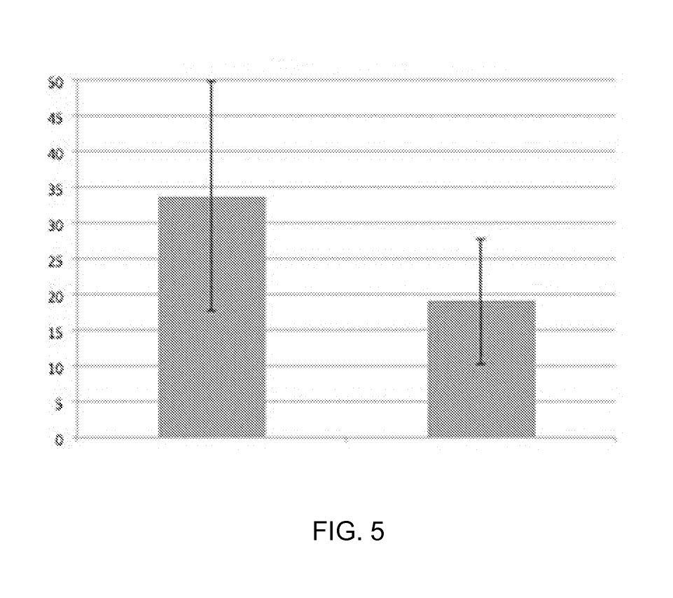

Detection of ORF-1 levels may be used to clarify the diagnosis of potential prostate cancer patients with PSA levels in the “grey zone.” FIG. 3 is a standard curve for ELISA measurements of ORF-1 in serum. Seven standards ranging from 0-20 ng/mL are used to construct the curve. FIGS. 4 and 5 are charts showing average ORF-1 concentrations in the serum of two populations of patients with PSA levels in the “grey zone.” One population of “grey zone” patients had a diagnosis of prostate cancer confirmed by biopsy.

A second population of “grey zone” patients had a diagnosis of no prostate cancer confirmed without biopsy (non-biopsy patents). Analysis was performed by a competitive ELISA assay similar to that in Example 1, including the patient samples in the same ELISA run as test samples. As shown in FIGS. 4 and 5, patients confirmed with prostate cancer had significantly higher ORF-1 levels than non-biopsy patients without prostate cancer.

Various aspects of different embodiments of the present invention are expressed in paragraphs X1, X2, X3, and X4, as follows:

X1. One aspect of the present invention pertains to a method for diagnosing LINE-1 overexpression-linked cancer. The method preferably includes determining the level of ORF-1 protein in a biological sample from a human patient suspected of having cancer, wherein elevated levels of ORF-1 protein relative to a normal control is indicative of cancer.

X2. Another aspect of the present invention pertains to a kit comprising at least one specific binding agent for ORF-1 protein and auxiliary reagents for measurement of ORF-1 protein in at least one of a plasma sample and a serum sample from a human patient.

X3. Yet another aspect of the present invention pertains to a method for the diagnosis of cancer. The method preferably includes providing a liquid sample obtained from a human. The method preferably includes directly contacting the liquid sample with an antibody specific for ORF-1 protein under conditions whereby a complex is formed between the antibody and ORF-1. The method preferably includes measuring the amount of complex formed. The method preferably includes comparing the amount of complex formed to a control amount determinative of the diagnosis of cancer.

X4. Still another aspect of the present invention pertains to a method of detecting prostate cancer. The method preferably includes providing a biological sample obtained from a human. The method preferably includes directly contacting the biological sample with an antibody specific for ORF-1 protein under conditions whereby a complex is formed between the antibody and ORF-1. The method preferably includes measuring the amount of complex formed. The method preferably includes comparing the amount of complex formed to a control amount determinative of the diagnosis of prostate cancer.

Yet other embodiments pertain to any of the previous statements X1, X2, X3 or X4 which are combined with one or more of the following other aspects.

Wherein the biological sample is a liquid sample.

Wherein the liquid sample is one of plasma, serum, and urine.

Wherein the liquid sample is one of plasma and serum.

Wherein determining the level of ORF-1 protein is performed using an immunoassay.

Wherein the determining the level of ORF-1 protein includes measuring the binding of ORF-1 to a specific binding agent for ORF-1.

Wherein the specific binding agent for ORF-1 is an antibody specific for ORF-1.

Wherein the antibody is configured to bind at least one of SEQ ID NO. 1, SEQ ID NO. 2, SEQ ID NO. 4, SEQ ID NO. 4, SEQ ID NO. 5, SEQ ID NO. 6, and SEQ ID NO. 7.

Which further comprises instructions for comparing a measured concentration of ORF-1 to specified predetermined concentrations of ORF-1 to determine risk of LINE-1 overexpression-linked cancer in the human patient.

Wherein the at least one specific binding agent for ORF-1 protein is an antibody specific for ORF-1 protein.

Wherein the antibody specific for ORF-1 protein is one of a monoclonal antibody and a polyclonal antibody.

Wherein the antibody specific for ORF-1 protein is configured to bind at least one of SEQ ID NO. 1, SEQ ID NO. 2, SEQ ID NO. 4, SEQ ID NO. 4, SEQ ID NO. 5, SEQ ID NO. 6, and SEQ ID NO. 7.

Wherein the biological sample is obtained from a human having a blood concentration of prostate-specific antigen between about 4 ng/ml and about 14 ng/ml.

Wherein the biological sample is one of serum, plasma, and urine.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1. A method for the diagnosis of LINE-1 overexpression-linked cancer, the method comprising determining the level of ORF-1 protein in a biological sample from a human patient suspected of having cancer, wherein elevated levels of ORF-1 protein relative to a normal control is indicative of cancer.

2. The method of claim 1, wherein the biological sample is a liquid sample.

3. The method of claim 2, wherein the liquid sample is one of plasma, serum, and urine.

4. The method of claim 3, wherein the liquid sample is one of plasma and serum.

5. The method of claim 1, wherein the determining the level of ORF-1 protein is performed using an immunoassay.

6. The method of claim 1, wherein the determining the level of ORF-1 protein includes measuring the binding of ORF-1 to a specific binding agent for ORF-1.

7. The method of claim 6, wherein the specific binding agent for ORF-1 is an antibody specific for ORF-1.

8. The method of claim 7, wherein the antibody is configured to bind at least one of SEQ ID NO. 1, SEQ ID NO. 2, SEQ ID NO. 4, SEQ ID NO. 4, SEQ ID NO. 5, SEQ ID NO. 6, and SEQ ID NO. 7.

9. A method for the diagnosis of cancer comprising:
a) providing a liquid sample obtained from a human;
b) directly contacting the liquid sample with an antibody specific for ORF-1 protein under conditions whereby a complex is formed between the antibody and ORF-1;
c) measuring the amount of complex formed; and
d) comparing the amount of complex formed to a control amount determinative of the diagnosis of cancer.

10. The method of claim 9, wherein the liquid sample is one of serum, plasma, and urine.

11. The method of claim 9, wherein the antibody specific for ORF-1 protein is one of a monoclonal antibody and a polyclonal antibody.

12. The method of claim 9, wherein the antibody specific for ORF-1 protein is configured to bind at least one of SEQ ID NO. 1, SEQ ID NO. 2, SEQ ID NO. 4, SEQ ID NO. 4, SEQ ID NO. 5, SEQ ID NO. 6, and SEQ ID NO. 7.

13. A method of detecting prostate cancer comprising:
a) providing a biological sample obtained from a human;
b) directly contacting the biological sample with an antibody specific for ORF-1 protein under conditions whereby a complex is formed between the antibody and ORF-1;
c) measuring the amount of complex formed; and
d) comparing the amount of complex formed to a control amount determinative of the diagnosis of prostate cancer.

14. The method of detecting prostate cancer of claim 13, wherein the biological sample is obtained from a human having a blood concentration of prostate-specific antigen between about 4 ng/ml and about 14 ng/ml.

15. The method of claim 13, wherein the biological sample is one of serum, plasma, and urine.

16. A kit comprising at least one specific binding agent for ORF-1 protein and auxiliary reagents for measurement of ORF-1 protein in at least one of a plasma sample and a serum sample from a human patient.

17. The kit of claim 16 further comprising instructions for comparing a measured concentration of ORF-1 to specified predetermined concentrations of ORF-1 to determine risk of LINE-1 overexpression-linked cancer in the human patient.

18. The kit of claim 16, wherein the at least one specific binding agent for ORF-1 protein is an antibody specific for ORF-1 protein.
